(19) United States
(12) Patent Application Publication
(10) Pub. No.: US 2006/0292185 A1
(43) Pub. Date: Dec. 28, 2006

(54) Topical skin preparations for treatment of skin aging comprising a testosterone ester

(76) Inventor: Shabtay Dikstein, Jerusalem (IL)

Correspondence Address: NATH & ASSOCIATES, 112 South West Street, Alexandria, VA, 22314, US

(21) Appl. No.: 10/568,275

(22) PCT Filed: Aug. 16, 2004
(86) PCT No.: PCT/IL04/00747

§ 371 Date: May 4, 2006

(30) Foreign Application Priority Data
Aug. 21, 2003 (IL) 157535

(51) Int. Cl. A61K 31/57; A61K 9/00

(52) U.S. Cl. 424/400; 514/178

(57) ABSTRACT

The present invention concerns Topical skin preparations for the use in the prevention of atrophy and aging of the skin, comprising a testosterone ester with an esterifying acid having between six to eleven carbon atoms.

### FIELD OF THE INVENTION

The present invention relates generally to the field of topical preparations for skin treatment. More specifically, the present invention relates to topical preparations for treatment of skin aging and atrophy.

### BACKGROUND OF THE INVENTION

When women enter into postmenopausal age, their estrogen as well as their androgen production drops significantly. Testosterone production in premenopausal women is about 250 micrograms per day whereas in postmenopausal women it is about 100 micrograms per day. In comparison to this, male testosterone production is about 7 milligrams per day. It is now recognized that postmenopausal women can have good benefits from both androgen and estrogen supplements. There are available on the market orally administered pills for postmenopausal women that contain orally active androgen methyltestosterone in addition to the usual estrogen.

It is known that androgens applied locally to the skin can have certain benefits. Estrogens are ineffective to the skin whereas corticosteroids accentuated degradative changes (Papa, C. M., J. Soc. Cosm. Chem. 18:549, 1967). However, the challenge has been to find an androgen derivative that is active locally but without any systemic influence. The absorption of testosterone through the skin is known, in fact, certain testosterone-delivering patches, worn on the skin, take advantage of this phenomena.

Thus, it would be desirable to have an androgen-containing preparation effective for diminishing the results of aging on the skin but without producing any side effects due to absorption into the bloodstream.

### SUMMARY OF THE INVENTION

Surprisingly, androgen-containing topical preparations that decrease atrophy of the skin due to aging or external factors, such as photoaging, or prolonged corticosteroid treatment have been found by the present invention.

Specifically, it has been found that locally applied testosterone esters (especially testosterone phenyl propionate), in which the ester potion contains more than 5 carbons, provided excellent local skin activity at relatively low concentrations. At such low concentrations, the absorption into the bloodstream is substantially negligible. Furthermore decrease in the absorption into the blood stream can be achieved by choosing the appropriate carrier in the form of an oil or a suspension.

The topical preparations of the present invention have been clinically shown to ameliorate the degradative changes of senescence or of external factors such as photodamage or corticosteroid atrophy, with negligible absorption into the blood. The toxicity of the compound of the invention has also been clinically determined.

It is noted that aging of the skin is mainly a problem of the dermis and by legal definition, a cosmetic product should not affect the dermis.

The present invention comprises topical skin preparation comprising a carrier and as the active ingredient testosterone ester including an esterifying acid having between six to eleven carbon atoms provided that the topical preparation has no estrogen or estrogen derivatives.

The present invention further relates to topical skin preparations for the treatment of atrophy and aging of the skin, comprising a carrier and as an active testosterone ester including an esterifying acid having between six to eleven carbon atoms provided that the topical preparation has no estrogen or estrogen derivatives.

The compounds of the invention should not contain any agent which is an estrogen or contain an estrogen derivative.

The present invention further concerns use of a testosterone ester including an esterifying acid having between six to eleven carbon atoms for the preparation of a topical skin preparation that does not contain estrogen or estrogen derivatives.

The present invention further concerns use of a testosterone ester including an esterifying acid having between six to eleven carbon atoms for the preparation of a topical skin preparation for the treatment of atrophy and aging of the skin, said preparation that does not contain estrogen or estrogen derivatives.

The present invention further concerns a method for treatment of atrophy or aging of the skin comprising administering to the skin an effective amount of a testosterone ester including an esterifying acid having between six to eleven carbon atoms.

The term “treatment”: refers to ameliorating, preventing or improving at least one parameter associated with atrophy and aging of the skin such as decrease in wrinkles (numbers, severity area), improved tonality, increase skin layers, improvement of components naturally reduced during aging such as collagen GAGs etc.

The topical compositions of the invention may be cosmetic or pharmaceutical compositions.

Examples of estrogen derivatives that should not be included in the preparation of the invention are estradiol or its esthers, estriol or its esters and the like.

Examples of testosterone esters including an esterifying acid are shown in Table 1 below.

TABLE 1
Testosterone ester esterifying acid of the invention

| Chemical Name | Number of carbon atoms | Case No. |
|---|---|---|
| Testosterone phenylpropionate | (C₉) | 1255-49-8 |
| Testosterone isocaproate | (C₆) | 15262-86-9 |
| Testosterone 17β-cypionate | (C₈) | 50-20-8 |
| Testosterone enanthate | CT | 315-37-7 |
| Testosterone undecanoate | (C₁₁) | 5949-44-0 |
| Testosterone decanoate | (C₁₀) | 5721-91-5 |
| Testosterone benzoate | (C₇) | 2088-71-3 |

According to preferred embodiments of the present invention, the testosterone ester is testosterone phenyl propionate (C9).

Preferably, the testosterone phenyl propionate is in a concentration of 0.1-3% w/w by weight of the total preparation, more preferably 0.5-2.5% w/w; most preferably 1% w/w.

Further according to preferred embodiments of the present invention, the skin preparations are the form of an oil solution. By another option they are in the form of a suspension. Preferably, the oil is a medium chain triglyceride. When choosing the carrier, it should be chosen so as to minimize, or eliminate absorption and to ensure stability.

Further according to preferred embodiments of the present invention the topical skin preparations may further include at least one additional therapeutically active agent, having no estrogenic activity. Examples of such agents are oil soluble vitamins or their derivatives such as vitamin A or retinoic acid, Vitamin D₃ or calcitiol, vitamin E, essential fatty acids or their esters.

### BRIEF DESCRIPTION OF THE INVENTION

The following are meant to provide non-limiting examples for preferred embodiments of the topical skin preparations of the present invention.

### EXAMPLES

### Example 1

### Oily Solution

Testosterone phenyl propionate 0.3% or 1% stirred in medium chain triglycerides, with slight heating to 40° C.-50° C. until totally dissolved.

Suitable preservatives, antioxidants, and/or perfumes may also be added.

### Example 2

### Oily Solution with Vitamin E

The preparation of this solution was carried out as in Example 1 above with the addition of 2% vitamin E.

### Example 3

### Oily Solution with Vitamin D

The preparation of this solution was carried out as in Example 1 above with the addition of 0.01% vitamin D₃.

### Example 4

### Oily Solution with Vitamin A

The preparation of this solution was carried out as in Example 1 above with the addition of 0.25% vitamin A₁.

### Example 5

### Testosterone Phenyl Propionate in an oil rich in Essential Fatty Acids

Testosterone phenyl propionate 0.1 to 1% dissolved in safflower oil.

### Example 6

### Clinical Studies

Eight postmenopausal women were treated with the skin preparation of Example 1 daily for three months on the skin of the inner arm. Biopsies were taken from the treated skin and from adjacent non-treated skin. The biopsies were evaluated and described according to the criteria described in Timar, F. et al, Skin Res. Technol. 6:17, 2000 incorporated herein at its entirety

The results are given in Table 2 below.

TABLE 2

| | Epidermal | | Dermal | |
|---|---|---|---|---|
| | Thickness | | Interdigitation | Amount of |
| Name | (microns) | Cell Layers | Index | GAG (%) |

Changes from Control (data)

| | | | | |
|---|---|---|---|---|
| TP 1 | 8.7 ± 1.5 (*) | 0.18 ± 0.37 | 0.22 ± 0.07 (*) | 2.7 ± 0.8 (*) |
| TP 0.3 | 0.3 ± 5.7 | −0.2 ± 0.45 | 0.07 ± 0.04 | 2.2 ± 1.3 |

GAG = glucose amino glycan
TP = Testosterone phenyl propionate
(*) = significantly different from normal old cohort
Interdigitation Index - was determined as in Timar et al., incorporated herein by reference.

### Example 7

### Pharmaokinetics Study

The preparation of Example 1 (0.3% TPP in oil) was prepared according to the procedure of example 1.

Test persons numbers: in the first period ((a) bellow)1 tested person, in the second ((b) bellow)₃ tested healthy postmenopausal female volunteers.

Test area: 300 cm² healthy skin in the middle of the back.

Treatment and sampling:
(a) single application of the 0.3% TPP oil in one person; blood samples were taken five times: before the treatment, after in 2, 4, 8 and 24 hours;
(b) repeated application of the 0.3% TPP oil six days each morning in succession in three persons; blood samples were taken five times:

before treatment, and after the first application in the 24, 72 hours, first and seventh days after the cessation of the treatment.

Analysis: by ELISA method the following parameters were measured: bounded testosterone, free testosterone, DEA sulfate, androstene dione, cortisol.

The values were expressed in pg/ml, ug/ml and ng/ml units.

Results

The level of the measured hormones, including the testosterone values corresponded to the normal circadian amounts after the single application of the 0.3% TPP oil showing that the preparation of the invention had no effect on hormonal levels.

During the repeated application in three persons the hormone levels altered in different directions—some increased and some decreased—, but the alternations remained in the normal range (see Table 3 below).

These results indicate little or no absorption of the compound of the invention both in single and repeated use.

TABLE 3

| | Sampling | | | | | Normal |
|---|---|---|---|---|---|---|
| Hormone | 1 | 2 | 3 | 4 | 5 | range |

Experiment (a)

| | | | | | | |
|---|---|---|---|---|---|---|
| Testosterone (pg/ml) | 231 | 233 | 316 | 304 | 300 | 200-700 |
| Free testosterone (pg/ml) | 5.13 | 5.42 | 7.35 | 7.14 | 6.78 | <12 |
| DEA sulfate (ug/ml) | 0.18 | 0.19 | 0.14 | 0.20 | 0.19 | 0.8-2.6 |
| Androstene dione (ng/ml | 0.68 | 0.45 | 0.70 | 0.62 | 0.68 | 1.0-3.0 |
| Cortisol (ng/ml) | 141 | 77 | 163 | 128 | 167 | 60-150 |

Experiment (b)

| | | | | | | |
|---|---|---|---|---|---|---|
| Testosterone (pg/ml) | 1. | 578 | 466 | 498 | 530 | 511 |
| | 2. | 463 | 471 | 486 | 606 | 529 |
| | 3. | 376 | 384 | 435 | 384 | 385 |
| Free testosterone (pg/ml) | 1. | 9.83 | 8.31 | 8.65 | 9.23 | 8.78 |
| | 2. | 8.42 | 8.37 | 8.56 | 9.87 | 9.25 |
| | 3. | 9.63 | 9.37 | 10.92 | 9.16 | 9.69 |
| DEA sulfate (ug/ml) | 1. | 0.41 | 0.38 | 0.50 | 0.50 | 0.39 |
| | 2. | 1.64 | 1.10 | 1.10 | 1.13 | 1.10 |
| | 3. | 0.12 | 0.14 | 0.12 | 0.08 | 0.11 |
| Androstene dione (ng/ml) | 1. | 0.76 | 0.69 | 0.77 | 1.27 | 1.08 |
| | 2. | n.d. | n.d. | n.d. | n.d. | n.d. |
| | 3. | 0.61 | 0.55 | 0.59 | 0.40 | n.d. |
| Cortisol (ng/ml) | 1. | 94 | 67 | 88 | 107 | 132 |
| | 2. | 228 | 84 | 138 | 109 | 184 |
| | 3. | 129 | 86 | 94 | 55 | 73 |

1. Topical skin preparation for women comprising a testosterone ester with an esterifying acid having between six to eleven carbon atoms, provided that the topical preparation does not comprise estrogen or estrogen derivatives.

2. Topical skin preparations for use by women for the prevention of atrophy and aging of the skin, comprising a testosterone ester with an esterifying acid having between six to eleven carbon atoms, provided that the topical preparation does not comprise estrogen or estrogen derivatives.

3. Topical skin preparations according to claim 1, wherein the testosterone ester is testosterone phenyl propionate.

4. Topical skin preparations according to claim 2, wherein the testosterone phenyl propionate is in a concentration of 0.1-3% by weight of the total preparation.

5. Topical skin preparations according to claim 4, wherein the testosterone phenyl propionate is in a concentration of 0.5% to 2.5% by weight of the total preparation.

6. Topical skin preparation according to claim 5, wherein the testosterone phenyl propionate is in a concentration of 1% by weight of the total preparation.

7. Topical skin preparations according to claim 2, in the form of an oil solution.

8. Topical skin preparations according to claim 7, wherein the oil is a medium chain triglyceride.

9. Topical skin preparations according to claim 1, further comprising at least one additional therapeutically active agent.

10. Topical skin preparations according to claim 9 wherein the additional therapeutic agent is selected from the group consisting of: vitamin A, retinoic acid, vitamin D₃, calcitriol, vitamin E, essential fatty acid, essential fatty acid ester, or a combination of at least two of the above agents.

11. Use of a testosterone ester with an esterifying acid having between six to eleven carbon atoms for the preparation of a topical skin preparation for women, provided that the topical preparation does not comprise estrogen or estrogen derivatives.

12. Use according to claim 11 wherein the topical skin preparations are for the prevention of atrophy and aging of the skin.

13. Use according to claim 11, wherein the testosterone ester is testosterone phenyl propionate.

14. Use according to claim 11, wherein the testosterone phenyl propionate is in a concentration of 0.1-3% by weight of the total preparation.

15. Use according to claim 14, wherein the testosterone phenyl propionate is in a concentration of 0.5% to 2.5% by weight of the total preparation.

16. Use according to claim 15, wherein the testosterone phenyl propionate is in a concentration of 1% by weight of the total preparation.

17. Use according to claim 11, wherein the topical skin preparation is in the form of an oil solution.

18. Use according to claim 17, wherein the oil is a medium chain triglyceride.

19. Use according to claim 11, wherein the topical preparation further comprises at least one additional therapeutically active agent.

20. Use according to claim 19 wherein the additional therapeutic agent is selected from the group consisting of: vitamin A, retinoic acid, vitamin D₃, calcitriol, vitamin E, essential fatty acid, essential fatty acid ester, or a combination of at least two of the above agents.

21. A method for the treatment of atrophy or aging of the skin of women comprising administering to a patient in need of such treatment a topical skin preparation as described in claim 1.
